(19) United States
(12) Patent Application Publication
(10) Pub. No.: US 2010/0111874 A1
(43) Pub. Date: May 6, 2010

(54) METHOD OF CANCER DETECTION AND TREATMENT

(75) Inventors: Leroy F. Liu, Bridgewater, NJ (US); Shyamal Desai, Somerset, NJ (US); Johnson Yin-Nam Lau, Newport Beach, CA (US)

Correspondence Address: FOX ROTHSCHILD LLP;PRINCETON PIKE CORPORATE CENTER, 997 LENOX DRIVE, BLDG. #3, LAWRENCEVILLE, NJ, 08648, US

(73) Assignee: University of Medicine and Dentistry of New Jresey, Somerset, NJ (US)

(21) Appl. No.: 12/532,375

(22) PCT Filed: Mar. 17, 2008
(86) PCT No.: PCT/US08/03509

§ 371 Date: Sep. 21, 2009

Related U.S. Application Data
(60) Provisional application No. 60/918,733, filed on Mar. 19, 2007.

(51) Int. Cl. A61K 49/00; A61K 31/203; A61K 38/21; A61K 38/19; A61K 31/4375; C12Q 1/68; G01N 33/53; G01N 33/574; A61P 35/00

(52) U.S. Cl. 424/9.2; 514/559; 424/85.4; 424/85.1; 514/283; 435/6; 435/7.21; 435/7.23

(57) ABSTRACT

The present invention provides methods for detecting and treating cancer. In some embodiments, levels of ISG15 are determined, and topoisomerase I and II inhibitors as well as other DNA-damaging agents plus agents that increase the expression of ISG15 are selected and administered.

### CROSS-REFERENCE TO RELATED APPLICATIONS

This application claims benefit of priority to U.S. Provisional Application No. 60/918,733, filed on Mar. 19, 2007, the entire disclosure of which is incorporated herein by reference.

### BACKGROUND OF THE INVENTION

Cancer is one of the leading causes of death in the world. Many treatment modalities exist, including chemotherapy. Chemotherapeutic agents include, for example, alkylating agents, antimetabolites, topoisomerase I and II inhibitors/poisons, and DNA-intercalating agents, among others. Various types of cancer respond to the same treatment in different ways. In particular, response to topoisomerase I inhibitors varies widely, both in vitro and in vivo.

Two camptothecin (CPT) analogs, irintotecan (Camptosar®) and topotecan (Hycamtin®), have been developed for the treatment of certain human cancers, including ovarian and colorectal cancers. However, not all cancers are sensitive to CPT-based chemotherapy. It is of particular significance that in vitro, CPT sensitivity varies greatly in a panel of breast and colon cancer cell lines. To date, the factors affecting tumor sensitivity to CPT are not well understood.

CPTs kill tumor cells by trapping topoisomerase I (TOP1)-DNA covalent complexes. The repair of this unique type of DNA damage, which is not fully understood, is expected to affect CPT sensitivity/resistance. Recent studies have demonstrated that an ubiquitin/26S proteasome pathway is activated by CPT leading to the degradation of TOP1-DNA covalent complexes in CPT-treated cells. It has been suggested that proteasomal degradation of TOP1 (TOP1 down-regulation) represents a repair mechanism for CPT-induced TOP1-DNA covalent complexes. Indeed, cells proficient in this repair process are more resistant to CPT. In addition, overexpression of Cullin3, a component of an SCF complex, has been shown to increase ubiquitination and TOP1 down-regulation, resulting in CPT resistance. Furthermore, co-treatment with the proteasome inhibitor, MG132, that inhibits TOP1 down-regulation, increases tumor cell sensitivity to CPT. These results suggest that the ubiquitin/proteasome pathway could be an important determinant for CPT sensitivity/resistance.

Several studies have demonstrated that CPT-induced TOP1 down-regulation is defective in many tumors, a factor which could contribute to tumor-specific killing by CPT. In tissue culture models, CPT has been shown to induce TOP1 down-regulation in normal non-transformed cells, but not in many tumor cells. In a nude mouse model, topotecan treatment has been shown to cause TOP1 down-regulation in many normal tissues, for example, blood, brain, kidney, liver and skin, but not in xenografted MDA-MB-435 breast cancer. Furthermore, patients receiving topotecan therapy also exhibit reduced TOP1 levels in normal peripheral blood cells, but not in leukemic cells. Thus, TOP1 degradation in normal tissues has been suggested to be a cellular response to evade the toxic effect of TOP1-directed anticancer drugs. Most tumor cells are defective in CPT-induced TOP1 degradation, which could explain in part the tumor cell sensitivity to CPTs.

The molecular basis for the defective proteasomal degradation of TOP1 in many tumor cells is not clear. However, recent studies have shown that the ubiquitin-like protein, ISG15, (Interferon-Stimulated Gene 15), is greatly elevated in many tumors. In addition, elevated expression of ISG15 has been shown to interfere with the ubiquitin/26S proteasome pathway, leading to altered degradation of many cellular proteins. ISG15 is conjugated to its substrates in much the same way as ubiquitin, requiring E1, E2 and E3, all of which are induced by type I interferons. The E1 for ISG15, UBE1L, is specific for ISG15. However, the E2 (UbcH8) and E3s (Rsp5, Herc5 and Efp) for ISG15 are dual functional E2/E3, required for both protein ISGylation and ubiquitination. It has been suggested that ISGylation, which is elevated in many tumors, interferes with ubiquitination through substrate competition at the E2/E3 level.

The ISG15 conjugation pathway is a determinant for tumor cell sensitivity to CPT by antagonizing CPT-induced TOP1 down-regulation. By knocking down either ISG15 or UbcH8 (the major E2 for ISG15), applicants have shown that the elevated ISG15 pathway in breast cancer ZR-75-1 cells indeed conferred increased CPT sensitivity by interfering with TOP1 down-regulation. In addition, applicants have shown that ISG15 expression is significantly reduced in a number of tumor cells selected for CPT resistance. These results suggest that ISG15 and factors in its conjugation pathway could serve as tumor biomarkers for CPT sensitivity/resistance.

In some embodiments the present invention relates to the use of ISG15 as a tumor biomarker for cancer screening based on the level of circulating ISG15 as measure by various methods for the detection of ISG15. In some embodiments the resent invention also relates to the use of circulating ISG15 as a marker for response to treatment.

In some embodiments the present invention relates to a method of determining the efficacy of topoisomerase I inhibitors. In other embodiments the present invention relates to the use of ISG15 and its conjugates for predicting tumor cell sensitivity to topoisomerase I-targeting drugs, for example topotecan and irintotecan. In still other embodiments the present invention relates to the use of type 1 interferons, as well as tumor necrosis factors and all-trans retinoic acid in combination with topoisomerase inhibitors as a method to treat topoisomerase-I inhibitor resistant tumor cells. Further, in some embodiments this invention also relates to the use of ISG15 and the enzymes involved in its conjugation pathway for predicting tumor cell sensitivity to topoisomerase II-targeting drugs, other DNA damaging drugs/agents, cisplatin and other platinum drugs, as well as radiation therapy.

### SUMMARY OF THE INVENTION

One embodiment of the present invention is to provide a method for diagnosing cancer in a subject. The subject is an animal, preferably a mammal, most preferably a human being. The method of diagnosis comprises obtaining a sample from the subject, determining the level of ISG15 in the sample using an assay, and comparing the level of ISG15 with that of a standard, wherein the presence of elevated ISG15 is indicative of the presence of cancer.

Another embodiment of the present invention is to provide a method for determining the sensitivity of cancer to DNA-damaging agents, such as topoisomerase I inhibitors. The method of determining the sensitivity of the cancer comprises obtaining a sample from the subject, determining the ISG15 level in the sample using an assay, and comparing the level of ISG15 to that of a standard, wherein the presence of elevated ISG15 is indicative of the cancer's sensitivity to treatment with a DNA-damaging agent, such as topoisomerase I and II inhibitors, as well as platinum based drugs and irradiation therapy.

Yet another embodiment of the present invention is to provide a method of identifying an appropriate regimen for the treatment of cancer in a subject, comprising obtaining a sample from the subject, determining the ISG15 level in the sample using an assay, comparing the level of ISG15 to that of a standard, selecting DNA-damaging agents appropriate to the cancer, and optionally selecting additional chemotherapeutic agents for combination therapy. In particular DNA-damaging agents that inhibit topoisomerase I and chemotherapeutic agents that upregulate ISG15 to enhance the sensitivity of the cancer to chemotherapy are in view.

Still another embodiment of the present invention is to provide a method of monitoring a course of cancer treatment with chemotherapeutic agents, comprising the steps of obtaining a sample from the subject, determining the ISG15 level in the sample using an assay, comparing the level of ISG15 to that of a standard, administering at least one chemotherapeutic agent to the subject, obtaining a second sample from the subject, measuring in the second sample the ISG15 level, and comparing the ISG15 level of the first sample with that of the second sample to determine changes in the sensitivity of the cancer to the chemotherapeutic agents. One embodiment of this method includes a topoisomerase I inhibitor as at least one of the chemotherapeutic agents. In another embodiment of this method at least one of the chemotherapeutic agents is a small molecule or biological molecule that upregulates ISG15 expression. Preferred small molecules include all-trans retinoic acid. Preferred biological molecules include interferons, tumor necrosis factors and mixtures thereof.

The samples obtained from the subject may be blood samples, including serum, plasma, and whole blood and peripheral blood cells, and/or cancer tissue samples.

The assay for determining the level of ISG15 may be an immunoassay that detects the expression of ISG15 protein, for example using monoclonal antibodies or polyclonal antibodies against the ISG15 protein. The assay may also be a nucleic acid-based assay that detects the expression of messenger RNA. In addition, other assays for determining the level of ISG15 may detect other markers that correlate with the expression of ISG15.

Yet another embodiment of the present invention is to use the ISG15 conjugation pathways E1, E2 and E2s as a marker for the same functional use as tumor marker present in the blood sample and the tumor sample.

These methods may also differentiate between benign and malignant tumors.

A further embodiment of the present invention is to provide a method for determining when to cease treatment of the subject with the chemotherapeutic agents.

Yet another embodiment of the present invention is to provide a method for the treatment of cancer, comprising administering to a subject in need thereof a therapeutically effective amount of a topoisomerase I inhibitor and an agent that increases the expression of ISG15. This method is particularly useful in cases wherein the cancer expresses low levels of ISG15, but may also be used when the cancer expresses an elevated level of ISG15 in order to enhance the therapeutic effect. The agent that increases the expression of ISG15 may be a small molecule, such as all-trans retinoic acid, or a biological molecule, for example, an interferon or a tumor necrosis factor, or mixtures thereof.

It is also an embodiment of the present invention to provide a pharmaceutical composition for treating cancer, comprising a therapeutically effective amount of a topoisomerase I inhibitor, for example topotecan or irintotecan, and a therapeutically effective amount of an agent that increases the expression of ISG15, for example all-trans retinoic acid, or an interferon or a tumor necrosis factor.

Another object of the present invention is to provide a pharmaceutical composition for treating cancer, comprising a therapeutically effective amount of topoisomerase II inhibitor/poison, platinum-containing drug, and/or radiation treatment.

One embodiment of the invention is a method to screen for effective topoisomerase I inhibitors.

Yet another embodiment of the invention is drug discovery tools, for example, cell lines, transfectants and vectors.

### BRIEF DESCRIPTION OF THE DRAWINGS

FIG. 1 shows that breast cancer cells exhibit highly varied ISG15 expression, CPT sensitivity, and TOP1 down-regulation. Panel A is a graph showing CPT sensitivity. Panel B is a picture of a gel showing ISG15 expression. Panel C is a picture of a gel showing CPT-induced down-regulation of TOP1.

FIG. 2 shows that elevated ISG15 expression in tumor cells is inversely correlated with TOP1 down-regulation. Panels A-C are pictures of gels showing ISG15 expression and TOP1 down-regulation. ISG15 expression is shown in the right panels and CPT-induced TOP1 down-regulation is shown in the left panels.

FIG. 3 shows that shRNA-mediated down-regulation of ISG15 in breast cancer ZR-75-1 cells confers CPT resistance and restores CPT-induced TOP1 down-regulation. Panel A is a picture of a gel showing reduced ISG15 expression in ZR-75-1 cells expressing ISG15 shRNA. Panel B is a graph showing CPT sensitivity. Panel C is a picture of a gel showing CPT-induced TOP1 down-regulation.

FIG. 4 shows that shRNA-mediated down-regulation of UbcH8 in breast cancer ZR-75-1 cells confers CPT resistance and restores CPT-induced TOP1 down-regulation. Panel A is a picture of a gel showing the reduced UbcH8 protein level in ZR-75-1 cells expressing UbcH8 shRNA. Panel B is a picture of a gel showing that UbcH8 shRNA decreases ISG15 conjugation in ZR-75-1 cells. Panel C is a graph showing that Knocking down UbcH8 confers CPT resistance. Panels D-F are pictures of gels showing that CPT-induced TOP1 down-regulation is restored in ZR-75-1 cells expressing UbcH8 shRNA.

FIG. 5 shows reduced ISG15 expression in various tumor cells selected for high levels of CPT resistance. Panel A is a picture of a gel showing that expression of ISG15 is reduced in tumor cells selected for CPT resistance. Panel B is a table showing the IC50 values obtained by the MTT assay, and the resistance ratios.

FIG. 6 shows reduced ISG15 expression in breast cancer MCF-7 cells selected for low levels of CPT resistance. Panel A is a picture of a gel showing formation of cleavable complexes in MCF7 and MCF7/RC breast cancer cells. Panel B is a picture of a gel showing CPT-induced TOP1 down-regulation in MCF7 and MCF7/RC cells. Panel C is a picture of a gel showing the same membrane filter as in B stripped and re-probed with anti-ISG15 antibody. Panel D is a table of IC50 values determined by the MTT assay, and the resistance ratio.

FIG. 7 illustrates a proposed model for the role of protein ISGylation in CPT sensitivity/resistance.

FIG. 8 is a table showing CPT sensitivity for cells expressing different levels of ISG15.

### DETAILED DESCRIPTION OF THE INVENTION

Topoisomerase I inhibitors work by binding to and inhibiting the enzyme so as to allow the accumulation of enzyme-DNA cleavable complexes. Topoisomerase inhibitors in the camptothecin family include, for example, camptothecin, irintotecan, topotecan, belotecan, 9-aminocamptotecan, 10-aminocamptotecan, 12-aminocamptotecan, 10,11-methylenedioxycamptotecan, 9-nitrocamptotecan, 9-nitro-10-hydroxycamptotecan, 10-hydroxycamptotecan, 11-formylcamtotecan, 10-chlorocamptotecan, 10-methylcamptotecan, as well as the salt forms of the above. These inhibitors have been shown to be down regulated by the ubiquitin/S26 proteasome pathway, which is defective in many tumors leading to differential sensitivity of the tumors to these inhibitors. Interferon inducible gene product 15 (ISG15) has been shown to be elevated in patients with cancer. Elevated ISG15 has been shown to interfere with the ubiquitin/S26 proteasome pathway, presumably through competition for substrates common for both the ubiquitin and ISGylation.

Previous studies have demonstrated that ISG15 and its conjugates are highly elevated in many tumors. Most strikingly, the expression levels of ISG15 and its conjugates are highly variable in different tumor cells. It has also been documented that CPT sensitivity varies greatly in different tumor cell lines and no single cellular parameter has been shown to correlate with CPT sensitivity. Applicants have shown that the expression levels of ISG15 and its conjugates correlate with CPT sensitivity among several pairs of cancer cell lines (ZR-75-1 vs. BT474 breast cancer cells, HT-29 vs. KM-12 colorectal cancer cells, and U138G and T98G glioblastoma cells).

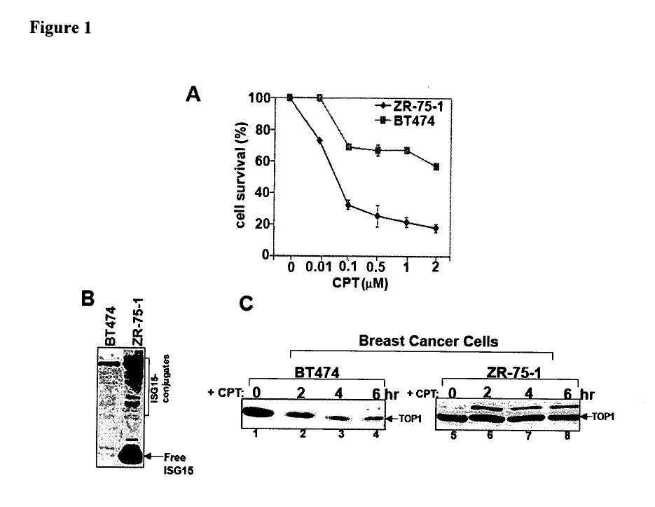

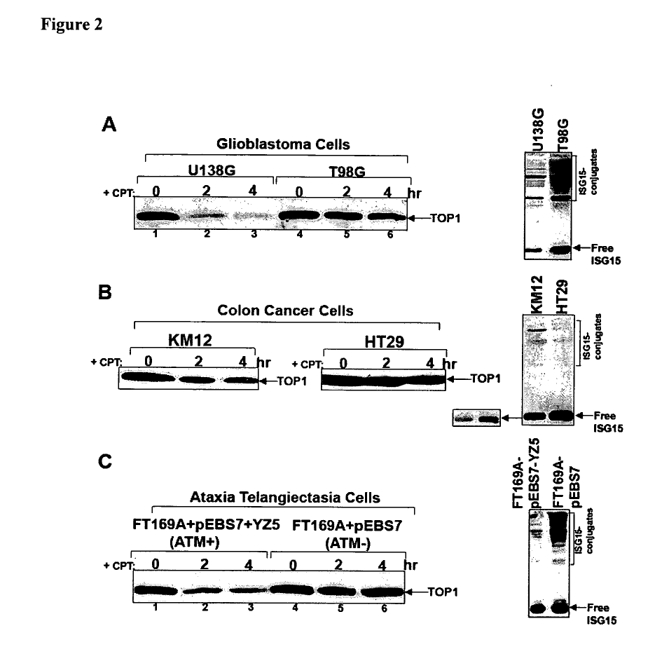

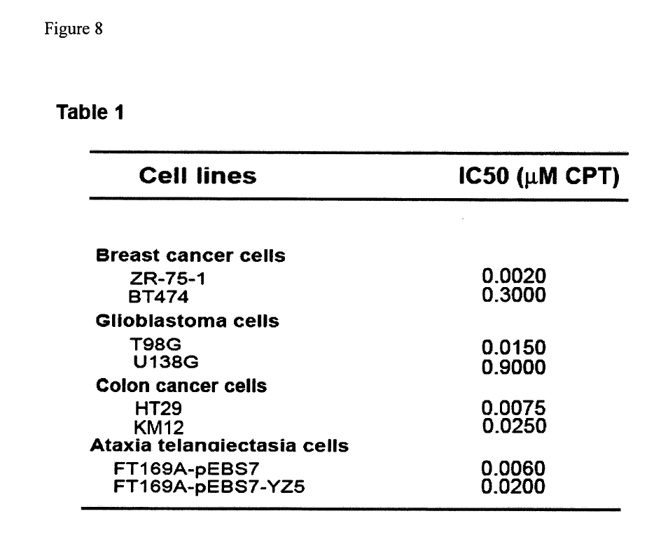

Breast cancer cells exhibit highly varied ISG15 expression, CPT sensitivity, and TOP1 down-regulation. CPT sensitivity has been shown to vary greatly among various breast cancer cells in vitro. No single parameter could account for the observed variation in CPT sensitivity. Applicants tested whether the expression levels of ISG15 and its conjugates may determine CPT sensitivity in breast cancer cells. As shown in FIG. 1A, ZR-75-1 cells are much more (greater than 50-fold based on IC50 values) sensitive to CPT than BT474 cells based on cell counting assay (one hr acute exposure). As shown in FIG. 1B, breast cancer ZR-75-1 cells express high levels of ISG15 and its conjugates while breast BT474 cells express very low levels of ISG15 and its conjugates. Additional cells, including glioblastoma (compare U138G and T98G glioblastoma cells), colorectal cancer cells (compare KM12 and HT29 colorectal cancer cells, see the underexposed film for ISG15 levels), and ATM+/ATM− cells, are also included in the comparison, and indeed, demonstrate an inverse correlation between ISG15 expression (FIG. 2, right panels) and CPT sensitivity (FIG. 8, Table 1, IC50s determined by MTT assay) among these cells.

Previous studies have shown that the ISG15 pathway interferes with the ubiquitin/26S proteasome pathway. It is possible that elevated expression of the ISG15 pathway in these cells could lead to increased CPT sensitivity by interfering with TOP1 down-regulation. Applicants measured CPT-induced degradation of TOP1 in these cells. As shown in FIG. 1C and FIG. 2 (left panels), cells expressing high levels of ISG15 are indeed much less efficient in TOP1 degradation. These results suggest the possibility that the ISG15 pathway may affect CPT sensitivity through its interference with TOP1 degradation.

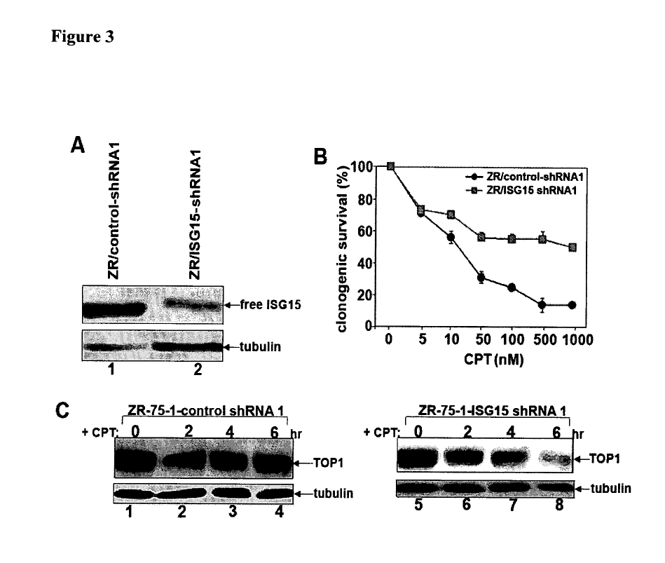

Elevated ISG15 expression in tumor cells leads to CPT sensitivity. In order to demonstrate a causal relationship between ISG15 expression and CPT sensitivity, shRNA-mediated knockdown of ISG15 in breast cancer ZR-75-1 cells was carried out. Several clones of ISG15 shRNA transfectants as well as those of control shRNA transfectants were isolated. One of the clones, ZR/ISG15-shRNA1 was extensively characterized. As shown in FIG. 3A, the expression level of ISG15 in ZR/ISG15-shRNA1 was significantly (more than 70%) reduced as compared to that in ZR/control-shRNA1. ZR/ISG15-shRNA1 cells were shown to be much more resistant to CPT (one hr acute exposure followed by cell survival measurement using a clonogenic assay) than control-shRNA cells (FIG. 3B), indicating that the high ISG15 levels in tumor cells may be causally responsible for their increased CPT sensitivity.

Applicants also investigated the possible mechanism for increased CPT sensitivity in ISG15 overexpressing tumors. As suggested from results shown in FIGS. 1 and 2, ISG15 overexpression is inversely correlated with TOP1 down-regulation. To test whether elevated ISG15 expression in tumor cells is causally responsible for reduced TOP1 down-regulation, TOP1 down-regulation is measured in both ZR/ISG15-shRNA1 and ZR/control-shRNA1 cells. As shown in FIG. 3C, similar to ZR-75-1 cells, ZR/control-shRNA1 cells exhibit minimal TOP1 down-regulation. By contrast, ZR/ISG15-shRNA1 cells exhibit greatly increased rate of TOP1 down-regulation (about 50% down-regulation in 2 hrs), suggesting that ISG15 negatively regulates TOP1 down-regulation. Together, these results suggest that reduced TOP1 down-regulation may contribute to increased CPT sensitivity in ISG15 overexpressing tumor cells.

Protein ISGylation in tumor cells alters CPT sensitivity. The above studies suggest that the elevated ISG15 protein level in tumor cells could contribute to CPT sensitivity by interfering with TOP1 degradation. However, previous studies have shown that protein ISGylation also interferes with protein polyubiquitination. Applicants therefore tested whether the formation of ISG15-protein conjugates (ISGylation) is responsible for increased CPT sensitivity in ZR-75-1 cells by knocking down the major E2, UbcH8, for ISG15. Several stable clones of UbcH8-shRNA transfected ZR-75-1 cells were isolated. Two clones, ZR/UbcH8-shRNA1 and ZR/UbcH8-shRNA2, were extensively characterized.

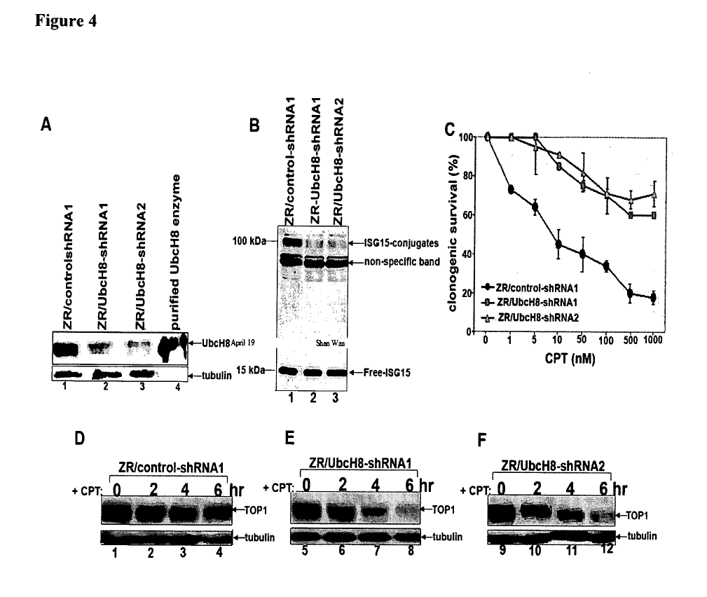

As shown in FIG. 4A, the expression levels of UbcH8 in ZR/UbcH8-shRNA1 and ZR/UbcH8-shRNA2 cells are reduced to 70 and 85%, respectively, as compared to that in ZR/control-shRNA2 cells (a different stable clone isolated from control shRNA-transfected ZR-75-1 cells). The levels of ISG15-protein conjugates in ZR/UbcH8-shRNA1 and ZR/UbcH8-shRNA2 cells are also significantly reduced as compared to that in control-shRNA2 cells. These results confirm the effectiveness of UbcH8 shRNA in knocking down UbcH8 in ZR-75-1 cells.

CPT sensitivity of ZR/UbcH8-shRNA1 and ZR/UbcH8-shRNA2 clones is measured by clonogenic survival following one-hr acute CPT exposure. As shown in FIG. 4C, both ZR/UbcH8-shRNA1 and ZR/UbcH8-shRNA2 are much more resistant to CPT than ZR/control-shRNA2, suggesting that the formation of ISG15-protein conjugates (ISGylation) affects CPT sensitivity.

Applicants also measured TOP1 down-regulation in these clones. Similar to ZR-75-1 cells, ZR/control-shRNA2 cells exhibited minimal TOP1 down-regulation (FIG. 4D). By contrast, ZR/UbcH8-shRNA1 (FIG. 4E) and ZR/UbcH8-shRNA2 (FIG. 4F) cells exhibit significantly increased rate of TOP1 down-regulation (about 50% degradation of TOP1 in 2 hrs). Together, these results suggest that the formation of ISG15-protein conjugates (ISGylation) in tumor cells interferes with TOP1 down-regulation and hence increases CPT sensitivity.

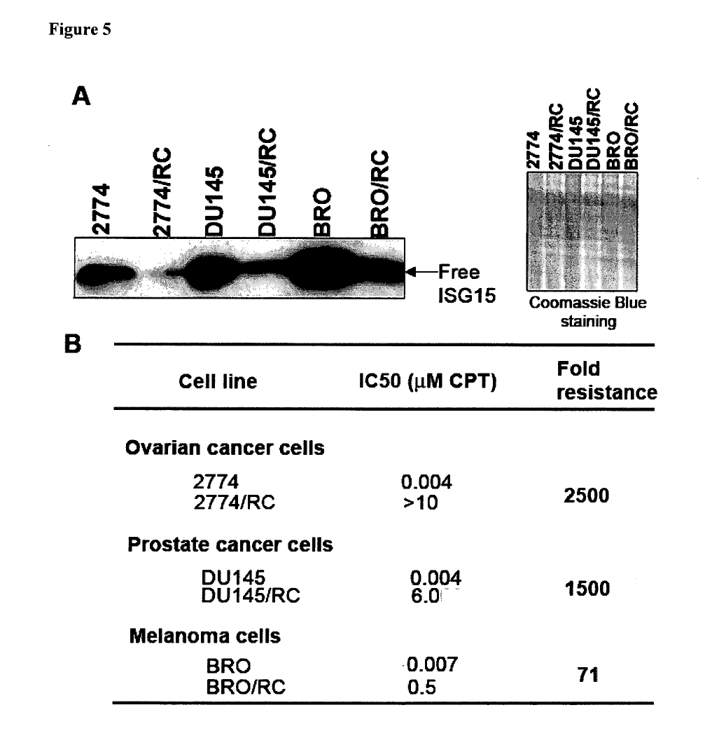

ISG15 expression is down-regulated in tumor cells selected for CPT resistance. Applicants' results have demonstrated that ISG15 and its conjugates are important determinants for intrinsic CPT sensitivity in various tumor cells. To test whether altered regulation of ISG15 may also contribute to acquired CPT resistance, the ISG15 protein levels are measured in three tumor cell lines selected for high levels of CPT resistance (see FIG. 5B for their CPT resistance ratios). As shown in FIG. 5A, ISG15 expression in these CPT-resistant cell lines (the ovarian cancer cell line 2774/RC, the prostate cancer cell line DU145/RC, and the melanoma cancer cell line Bro/RC) (see FIG. 5B their respective CPT resistance ratios) is greatly (more than five-fold) reduced as compared to that in their respective parental cells (2774, DU145, and Bro cells). The growth rates of these CPT-resistant cells are comparable to those of their respective parental cells (data not shown). These results suggest that ISG15 expression may play an important role in acquired CPT resistance.

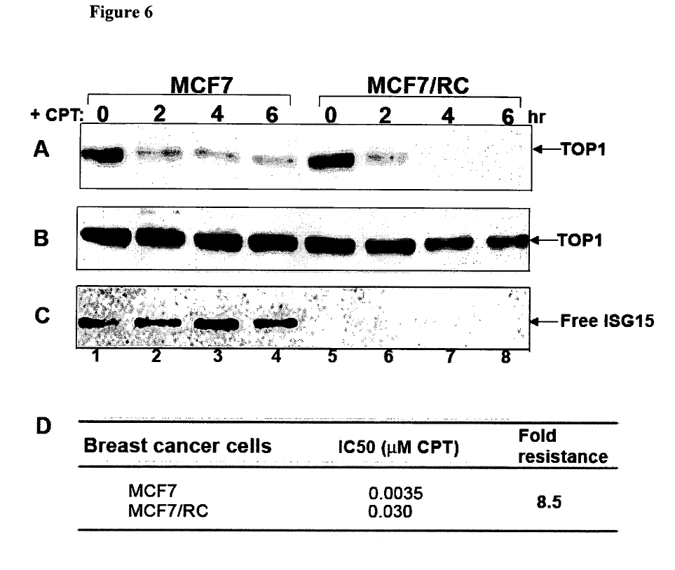

The resistant clones described above are selected for high levels of CPT resistance with stepwise increase in CPT concentrations. Many genetic changes are likely to have occurred in these resistant clones, which may complicate the interpretation of the role of ISG15 expression in CPT resistance. To avoid this problem, applicants also measure the ISG15 protein level in MCF7 breast cancer cells selected for low levels of CPT resistance. The low-level CPT resistance is also more likely to mimic clinical CPT resistance. As shown in FIG. 6B, MCF7/RC (IC50=0.030 μM), one of the low-level CPT-resistant clones of MCF7, is about 8-fold more resistant to CPT than MCF7 cells (IC50=0.0035 μM). No change in the amount of TOP1-DNA covalent complexes is observed (FIG. 6A, top panel). However, the ISG15 protein level in MCF7/RC is significantly (about 9-fold) reduced as compared to that in MCF7 cells (FIG. 6A, bottom panel). Interestingly, TOP1 down-regulation, which is minimal in the parental MCF7 cells, is increased in MCF7/RC cells (FIG. 6A, middle panel). Together, these results suggest that ISG15 expression is an important determinant for acquired CPT resistance, and provide additional support for the negative regulatory role of ISG15 in TOP1 down-regulation.

shRNA-mediated knockdown studies have further demonstrated that the expression level of the ISG15 conjugation pathway in tumor cells is indeed an important determinant for CPT sensitivity. This conclusion is based primarily on results from two studies; First, shRNA-mediated knockdown of ISG15 in ZR-75-1 breast cancer cells (known to express high levels of ISG15 and ISG15 conjugates) resulted in reduced CPT sensitivity. Second, shRNA-mediated knockdown of UbcH8 (the major E2 for ISG15) in ZR-75-1 cells also resulted in reduced CPT sensitivity. These results suggest that the formation of ISG15-protein conjugates (ISGylation) is responsible for reduced CPT sensitivity. However, a lower exposure of FIG. 4B also shows approximately 50% reduction of the free ISG15 level in UbcH8 shRNA knockdown cells (data not shown). Consequently, the possibility could not be ruled out that free ISG15 may also contribute to the interference with protein polyubiquitination and hence CPT sensitivity.

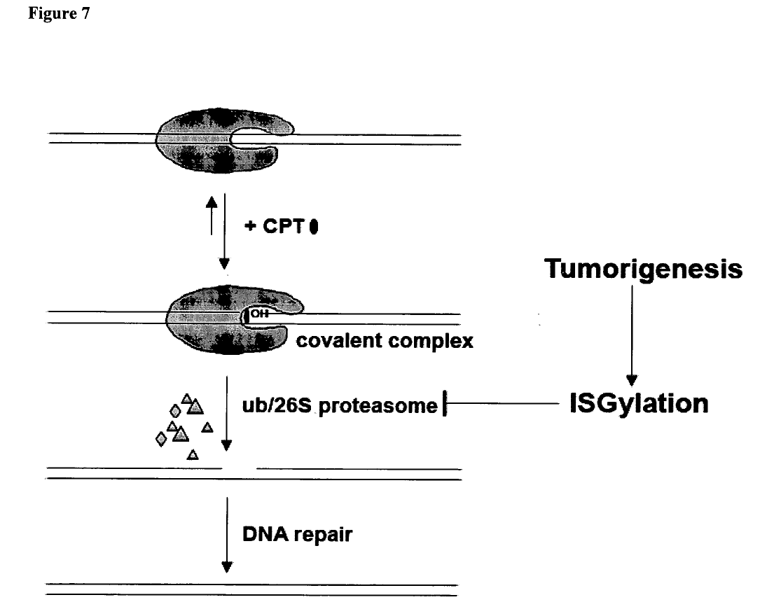

Previous studies have shown that shRNA-mediated knockdown of either ISG15 or UbcH8 results in increased protein polyubiquitination. It has been suggested that protein ISGylation interferes with protein polyubiquitination, leading to reduced degradation of many proteins. Since CPT-induced proteasomal degradation of TOP1 (TOP1 down-regulation) has been suggested to be a repair mechanism for CPT-induced DNA lesion, it seems reasonable to suggest that elevated protein ISGylation in tumor cells may inhibit CPT-induced TOP1 down-regulation, leading to increased CPT sensitivity. Indeed, applicants have shown that shRNA-mediated knockdown of either ISG15 or UbcH8 results in increased TOP1 down-regulation. Consequently, applicants propose that elevated expression of ISG15 and its conjugates in tumor cells inhibits CPT-induced TOP1 down-regulation, leading to increased CPT sensitivity as depicted in FIG. 7.

The importance of the ISG15 conjugation pathway in CPT sensitivity/resistance is further supported from studies of cell lines selected for acquired CPT resistance. Among five cell lines selected for high levels of CPT resistance, three of them (2774/RC ovarian cancer cells, DU145/RC prostate cancer cells and BRO/RC melanoma cells) show significant reduction of ISG15 expression as compared to their respective wild type cells. The other two CPT-resistant cell lines, SB1B/RC melanoma cells and U937/RC cells show no detectable ISG15. However, their respective parental cells, SB1B and U937 cells, also failed to show any detectable expression of ISG15. In addition to cells selected for high levels of CPT resistance, MCF7 breast cancer cells selected for a low level of CPT resistance (9-fold), which could be more clinically relevant, also show significant reduction of ISG15 expression. These results suggest that decreased ISG15 expression is an important mechanism for acquired CPT resistance in ISG15-overexpressing tumors.

While not wishing to be bound by any particular theory, applicants' results support the model shown in FIG. 7. In this model, CPT induces TOP1-DNA covalent complexes which are the key lesion responsible for tumor cell killing. Ubiquitin/26S proteasome-mediated degradation of TOP1-DNA covalent complexes represents a repair mechanism for cell survival. During tumorigenesis, the ISG15 conjugation pathway is elevated, which interferes with the ubiquitin/26S proteasome pathway leading to defective repair of TOP1-DNA covalent complexes and hence increased CPT sensitivity. The reason for elevated expression of the ISG15 pathway in tumors is currently being investigated.

Applicants' results have demonstrated a significant role of ISG15 in determining CPT sensitivity/resistance. These results have significant implications in the clinic for cancer patients. The highly variable expression of ISG15 and its conjugates in tumors can be used as predictors for cancer cell sensitivity to CPT. This is particularly important since both ISG15 expression levels and CPT sensitivity are highly variable among different tumors. It is also interesting to note that, in addition to being present in tumors, ISG15 has been shown to be secreted by tumor cells and detectable in blood. The blood ISG15 level may therefore be conveniently used for both diagnosis of cancer and prediction for CPT (and other TOP1-directed drugs) treatment response.

Modulation of the ISG15 pathway can have implications in drug combinations in the clinic. Since type I interferons are known to induce the ISG15 pathway, a combined use of type I interferons and CPT (or other TOP1-targeting drugs) is expected to increase CPT sensitivity in tumors expressing low levels of ISG15. Indeed, interferons have been shown to exhibit synergistic anticancer activity with CPT-11 against human colon cancer xenografts in nude mice. It is also interesting to note that all-trans retinoic acid is known to induce ISG15 expression, possibly through type I interferons. Consequently, all-trans retinoic acid, like type I interferons, can be used in combination with CPT (or other TOP1-targeting drugs) to sensitize tumors with low levels of ISG15 expression. The fact that the ISG15 pathway is down-regulated in many CPT-resistant tumor cells suggests that interferons, tumor necrosis factors or retinoic acid can be used in combination with CPT (or other TOP1 targeting drugs) to treat patients which have already developed CPT resistance.

In view of the above, one embodiment of the present invention is to provide a method for diagnosing cancer in a subject. The subject is an animal, preferably a mammal, most preferably a human being. The method of diagnosis comprises obtaining a sample from the subject, determining the level of ISG15 or E1/E2/E3 in the sample using an assay, and comparing the level of ISG15 with that of a standard, wherein the presence of elevated ISG15 or E1/E2/E3 is indicative of the presence of cancer. The standard ISG15 or E1/E2/E3 level would be that found in normal healthy subjects and/or subjects with non-cancerous diseases. The samples obtained from the subject may be blood samples, including serum, plasma, and whole blood, and/or cancer tissue samples.

Another embodiment of the present invention is to provide a method for determining the sensitivity of cancer to DNA-damaging agents. The method of determining the sensitivity of the cancer comprises obtaining a sample from the subject, determining the ISG15 or E1/E2/E3 level in the sample using an assay, and comparing the level of ISG15 or E1/E2/E3 to that of a standard, wherein the presence of elevated ISG15 or E1/E2/E3 is indicative of the cancer's sensitivity to treatment with a DNA-damaging agent. Preferred DNA-damaging agents are topoisomerase I inhibitors. The standard ISG15 level would be that found in other subjects with the same cancer, with or without knowledge of their response to DNA-damaging agents.

Yet another embodiment of the present invention is to provide a method of identifying an appropriate regimen for the treatment of cancer in a subject, comprising obtaining a sample from the subject, determining the ISG15 level in the sample using an assay, comparing the level of ISG15 to that of a standard, selecting DNA-damaging agents appropriate to the cancer, and optionally selecting additional chemotherapeutic agents for combination therapy. In particular DNA-damaging agents that inhibit topoisomerase I and chemotherapeutic agents that upregulate ISG15 to enhance the sensitivity of the cancer to chemotherapy are in view. Particularly preferred agents that upregulate ISG15 include all-trans retinoic acid, interferons, tumor necrosis factors, and mixtures thereof.

Still another embodiment of the present invention is to provide a method of monitoring a course of cancer treatment with chemotherapeutic agents, comprising the steps of obtaining a sample from the subject, determining the ISG15 level in the sample using an assay, comparing the level of ISG15 to that of a standard, administering at least one chemotherapeutic agent to the subject, obtaining a second sample from the subject, measuring in the second sample the ISG15 level, and comparing the ISG15 level of the first sample with that of the second sample to determine changes in the sensitivity of the cancer to the chemotherapeutic agents. A particularly preferred embodiment of this method includes a topoisomerase I inhibitor as at least one of the chemotherapeutic agents. In another embodiment of this method at least one of the chemotherapeutic agents is a small molecule or biological molecule that upregulates ISG15 expression. Preferred small molecules include all-trans retinoic acid. Preferred biological molecules include interferons, tumor necrosis factors and mixtures thereof.

The assay for determining the level of ISG15 may be an immunoassay that detects the expression of ISG15 protein, for example using monoclonal antibodies or polyclonal antibodies against the ISG15 protein. The assay may also be a nucleic acid-based assay that detects the expression of messenger RNA. In addition, other assays for determining the level of ISG15 may detect other markers that correlate with the expression of ISG15.

These methods may also differentiate between benign and malignant tumors.

A further embodiment of the present invention is to provide a method for determining when to cease treatment of the subject with the chemotherapeutic agents.

Yet another embodiment of the present invention is to provide a method for the treatment of cancer, comprising administering to a subject in need thereof a therapeutically effective amount of a topoisomerase I inhibitor and an agent that increases the expression of ISG15. This method is particularly useful in cases wherein the cancer expresses low levels of ISG15, but may also be used when the cancer expresses an elevated level of ISG15 in order to enhance the therapeutic effect. The agent that increases the expression of ISG15 may be a small molecule, such as all-trans retinoic acid, or a biological molecule, for example, an interferon or a tumor necrosis factor, or mixtures thereof.

Another embodiment of the present invention to provide a pharmaceutical composition for treating cancer, comprising a therapeutically effective amount of a topoisomerase I inhibitor and a therapeutically effective amount of an agent that increases the expression of ISG15, for example all-trans retinoic acid, an interferon, a tumor necrosis factor or mixtures thereof. The topoisomerase I inhibitor may be selected from the group consisting of camptothecin, irintotecan, topotecan, belotecan, 9-aminocamptotecan, 10-aminocamptotecan, 12-aminocamptotecan, 10,11-methylenedioxycamptotecan, 9-nitrocamptotecan, 9-nitro-10-hydroxycamptotecan, 10-hydroxycamptotecan, 11-formylcamtotecan, 10-chlorocamptotecan, 10-methylcamptotecan, and mixtures thereof.

One embodiment of the invention is a method to screen for effective topoisomerase I inhibitors.

Yet another embodiment of the invention is drug discovery tools, for example, cell lines, transfectants and vectors.

### Examples

Materials and Methods.

Cells. All cells are cultured in RPMI supplemented with 10% fetal bovine serum, L-glutamine (2 mM), penicillin (100 units/ml), and streptomycin (100 μg/ml) in a 37° C. incubator with 5% CO2. Breast cancer ZR-75-1 cells stably expressing ISG15 or UbcH8 shRNA are maintained in hygromycin B (100 μg/ml).

Immunoblotting of ISG15. Cells (5×105) are plated in 35×10 mm tissue culture dishes and incubated at 37° C. in a CO2 incubator. One day after plating, cells are lysed with 2× SDS gel sample buffer. After boiling for 10 min, cell lysates are sonicated and then subjected to analysis by 15% SDS-PAGE. Proteins are transferred onto nitrocellulose membrane. Immunoblotting of ISG15 is carried out using anti-ISG15 antibody following the enhanced chemiluminescence (ECL) Western procedure. The signal is detected using a Kodak Image Station 2000R.

Band depletion assay for TOP1 cleavage complexes. Cells (106/sample) are treated with CPT (25 μM in 1% DMSO) for various times at 37° C. Cells are then lysed with 0.2 N NaOH containing 2 mM EDTA as described previously (14). Cell lysates are then neutralized with 1/10 volume of a solution containing 10% NP-40, 1 M Tris (pH 7.4), 0.1 M MgCl2, 0.1 M CaCl2, 10 mM dithiothreitol, 1 mM EGTA, and a mixture of peptide protease inhibitors (100 μg/ml each of leupeptin, pepstatin, and aprotinin), followed by the addition of another 1/10 volume of 2 N HCl. Reactions are terminated by the addition of SDS-PAGE sample buffer. Immunoblotting analysis of cell lysates is carried out using anti-Scl-70 antibody from scleroderma patients as described above.

Immunoblotting assay for CPT-induced TOP1 degradation. Cells are prepared for immunoblotting as described above with two modifications. First, cells are incubated in CPT-free medium for 30 min prior to lysis (to reverse TOP1 cleavage complexes and to de-conjugate TOP1-ubiquitin and TOP1-UBL conjugates). Second, neutralized cell lysates are incubated with Staphylococcus aureus nuclease S7 (60 U/reaction) for 20 min on ice (to release TOP1 from residual TOP1-DNA covalent complexes) prior to termination with SDS-PAGE sample buffer.

Construction of ISG15 and UbcH8 shRNA vectors. ISG15 shRNA vector: The pSilencer 4.1 CMV-hygro shRNA expression vector (Ambion) is utilized for constructing ISG15-shRNA expression vector. Sense (5′-GATCCTGCGACGAACCTCTGAACATTCAAGAGATTACGCTGCTTGGAGACTTGTA-3′) (SEQ ID NO: 1) and antisense (5′AGCTTACAAGTCTCCAAGCAGCGTAATCTCTTGAATGTTCAGAGGTTCGTCGCAG-3′) (SEQ ID NO: 2) shRNA oligonucleotides, targeting the 232-250 region of ISG15 (accession no. AY168648), are synthesized (IDTDNA). The oligonucleotides are diluted in TE (10 mM Tris, 1 mM EDTA) to approximately 1 μg/ml. The sense and antisense oligonucleotides are then annealed by heating to 90° C. for 30 min in an annealing solution (provided by the manufacturer), followed by incubation at 37° C. for 1 hr. The annealed DNA is ligated into the pSilencer 4.1 CMV-hygro vector DNA cut with the BamHI and HindIII restriction enzymes as recommended by manufacturer, followed by transformation into E. coli. DH5α. Plasmid DNA is then purified and sequenced using the supplied primer sequences (5′AGGCGATTAAGTTGGGTA-3′ (SEQ ID NO: 3), 5′-CGGTAGGCGTGTACGGTG-3′ (SEQ ID NO: 4)) for verification. Ubch8 shRNA vector: The pSilencer 4.1 CMV-hygro shRNA expression vector (Ambion) is utilized for constructing the UbcH8-shRNA expression vector. Sense (5′-GATCCCGAGAACGGACAGATTTGCTTCAAGAGATTGCTCTTGCCTGTCTAAACGA-3′) (SEQ ID NO: 5) and antisense (5′-AGCTICGITTAGACAGGCAAGAGCAATCTCTTGAAGCAAATCTGTCCGTTCTCGG-3′) (SEQ ID NO: 6) shRNA oligonucleotides, targeting the 237-255 region of UbcH8 (accession no. AF031141), are synthesized (IDTDNA). Construction and verification of UbcH8-shRNA vector are carried out as described above. Control shRNA vector: As a negative control, the pSilencer 4.1 CMV-hygro Negative Control plasmid is utilized (Ambion). The plasmid encodes a shRNA whose sequence is not found in the mouse, human, or rat genome databases.

Construction of shRNA stable transfectants. Breast cancer ZR-75-1 cells (1×106) are transfected with 4 μg each of XMNI-linearized ISG15, UbcH8 or control shRNA vectors using the Polyfect transfection reagent (Qiagen). Following incubation for 72 hrs, cells are washed with fresh RPMI medium and incubated at 37° C. for 24 hrs. Cells are then trypsinized and replated into 150 mm tissue culture plates. Selection media containing 200 μg/ml hygromycin B is then added to the cells 24 hrs after replating. Individual colonies are picked following 3 weeks of hygromycin selection and screened for ISG15 and UbcH8 expression by Western blotting analysis using anti-ISG15 and anti-UbcH8 antisera, respectively.

Cell survival assays. MTT assay: Cells (3000 cells/well) are plated in 96-well microtiter plates and incubated at 37° C. in a CO2 incubator. For determination of IC50, cells are treated with varying concentrations of CPT for four days. Cells are then incubated with MTT (3-(4,5-dimethylthiazol-2-yl)-2,5-diphenyltetrazolium bromide) for 4 hrs at 37° C. The dye produced by viable cells is dissolved in DMSO and the optical density measured at 570 nm. All assays are performed at least twice in six replicate wells. Clonogenic assay: Cells (100-500 cells/plate) are plated in 6-well tissue culture plates and incubated at 37° C. in a CO2 incubator. Cells are then treated with various concentrations of CPT for 1 hr. Following CPT treatment, cells are washed free of CPT and placed in fresh medium. After 10 days, survived colonies are stained with Methylene blue and counted using the Minicount machine. Cell counting assay: Logarithmically growing cells are treated with various concentrations of CPT for 1 hr. CPT is then removed by washing the plates with CPT-free medium four times. Washed cells are allowed to grow in CPT-free medium for 4 days. Cell survival is determined by counting cells using the Coulter counter after trypsinization.

Example No. 1. FIG. 1 shows that breast cancer cells exhibit highly varied ISG15 expression, CPT sensitivity, and TOP1 down-regulation. Panel A, CPT sensitivity. Breast cancer ZR-75-1 and BT474 cells are treated with CPT for 1 hr followed by incubation in CPT-free medium for 3 days. Cell survival is determined by cell counting using a Coulter counter as described in Materials and Methods. Panel B, ISG15 expression. Lysates from semi-confluent breast cancer BT474 and ZR-75-1 cells (5×10⁵ each) are analyzed by 15% SDS-PAGE, followed by immunoblotting using anti-ISG15 antibody and ECL Western procedure (Pierce). Panel C. CPT-induced down-regulation of TOP1. Breast cancer BT474 and ZR-75-1 cells (10⁶ cells/sample) are treated with either 1% DMSO (lanes #1 and 5) or CPT (25 μM in 1% DMSO) (lanes #2-4 and 6-8) for various times at 37° C. Subsequently, cells are placed in fresh CPT-free tissue culture media for 30 min to reverse cleavage complexes. Cells are then lysed using the alkaline lysis procedure as described below. Neutralized lysates are further treated with Staphylococcus nuclease S7 (15 min on ice) to digest DNA and release TOP1 from TOP1-DNA covalent complexes. Reactions are terminated by addition of SDS-PAGE sample buffer. Immunoblotting is performed using anti-hTOP1 antisera obtained from scleroderma patients following the ECL Western procedure (Pierce).

Example No. 2. FIG. 2 shows that elevated ISG15 expression in tumor cells is inversely correlated with TOP1 down-regulation. Panels A-C. ISG15 expression and TOP1 down-regulation. Both ISG15 expression (right panels) and CPT-induced TOP1 down-regulation (left panels) are determined in glioblastoma U138G and T98G cells (10⁶/sample) (panel A), colorectal cancer HT29 and KM12 cells (panel B), and FT169A-pEBS7 (ATM−) and FT169A-pEBS7-YZ5 (ATM+) cells (panel C). Cells are treated with either 1% DMSO (lanes #1 and 4) or CPT (25 μM in 1% DMSO) (lanes #2, 3, 5 and 6) for various times at 37° C. Subsequently, cells are placed in fresh CPT-free tissue culture media for 30 min to reverse cleavage complexes. Cells are lysed and immunoblotted using either anti-hTOP1 (left panels) or anti-ISG15 (right panels) antisera as described in FIG. 1.

Example No. 3. FIG. 3 shows that shRNA-mediated down-regulation of ISG15 in breast cancer ZR-75-1 cells confers CPT resistance and restores CPT-induced TOP1 down-regulation. Panel A, Reduced ISG15 expression in ZR-75-1 cells expressing ISG15 shRNA. Cell lysates of ZR-75-1 clones expressing either control shRNA (lane #1) or ISG15 shRNA (lanes #2) are analyzed by 15% SDS-PAGE. Western blotting analysis is performed using anti-ISG15 antisera (upper panel). Equal protein loading is assessed by re-probing the same membrane with anti-tubulin antibody (lower panel). Panel B, CPT sensitivity. Clonogenic survival assay is carried out as described in Materials and Methods. Colonogenic survival curves of a ZR-75-1 clone expressing control shRNA and ISG15 shRNA after CPT treatment is shown. Mean±SD values from at least two experiments are presented. Panel C, CPT-induced TOP1 down-regulation. ZR-75-1 cells stably transfected with control shRNA (lanes 1-4) or ISG15 shRNA (lanes #5-8) (10⁶ cells/sample) are treated with either DMSO (lanes #1 and 5) or CPT (25 μM in 1% DMSO) (lanes #2-4 and 6-8) for various times at 37° C. Subsequently, cells are placed in fresh CPT-free media for 30 min to reverse cleavable complexes. Cell lysis and immunoblotting using anti-hTOP1 antisera are carried out as described in FIG. 1 (upper panels). Equal protein loading is assessed by re-probing the same membrane blot with anti-tubulin antibody (lower panels).

Example No. 4. FIG. 4 shows that shRNA-mediated down-regulation of UbcH8 in breast cancer ZR-75-1 cells confers CPT resistance and restores CPT-induced TOP1 down-regulation. Panel A, the reduced UbcH8 protein level in ZR-75-1 cells expressing UbcH8 shRNA. Cell lysates prepared from ZR-75-1 clones expressing either control shRNA (lane #1) or UbcH8 shRNA (lanes #2 and 3, from two independent clones), and the purified UbcH8 protein (lane #4) are analyzed by 15% SDS-PAGE. Western blotting analysis is performed using anti-UbcH8 antibody (Abeam). Equal protein loading is assessed by re-probing the same membrane with anti-tubulin antibody (lower panels). Panel B, UbcH8 shRNA decreases ISG15 conjugation in ZR-75-1 cells. Cell lysates prepared from ZR-75-1 clones expressing either control shRNA (lane #1) or UbcH8 shRNA (lanes #2 and 3, two independent clones), and the purified UbcH8 protein (lane #4) are analyzed by 15% SDS-PAGE. Western blotting analysis is performed using anti-ISG15 antibody. The non-specific bands are due to the crossreactivity of the ISG15 antibody to bovine serum albumin. Panel C, knocking down UbcH8 confers CPT resistance. ZR-75-1 clones expressing control shRNA and UbcH8 shRNA (two independent clones) are treated with CPT and cell survival is measured as described in Example 3. Panels D-F, CPT-induced TOP1 down-regulation is restored in ZR-75-1 cells expressing UbcH8 shRNA. Clonal cells of ZR-75-1 expressing control (panel D) or UbcH8 shRNA (panels E and F, representing two independent clones) (10⁶/sample) are treated with either DMSO (lanes #1, 5, and 9) or CPT (25 μM in 1% DMSO) (lanes #2-4, 6-8, and 10-12) for various times at 37° C. Cells are then placed in fresh CPT-free media for 30 min to reverse cleavage complexes. Cell lysis and immunoblotting using anti-hTOP1 antibody are carried out as described in Example 1.

Example No. 5. FIG. 5 shows reduced ISG15 expression in various tumor cells selected for high levels of CPT resistance. Panel A, expression of ISG15 is reduced in tumor cells selected for CPT resistance. The human ovarian cancer 2774 and its CPT-resistant variant 2774/RC (resistant to 1 μM CPT), the human prostate cancer DU145 and its CPT-resistant variant DU145/RC (resistant to 1 μM CPT) and melanoma Bro and its CPT-resistant variant Bro/RC (resistant to 100 nM CPT) cells are analyzed by 15% SDS-PAGE, followed by immunoblotting with anti-ISG15 antibody using the ECL Western procedure (Pierce) (left panel). A duplicate gel is stained with Coomassie blue to assess protein loading (right panel). Panel B, determination of IC50s by MTT assay. Cells are treated with different concentrations of CPT for 4 days. MTT assay is then performed as described in Materials and Methods. MTT assay for each CPT concentration is performed at least twice in six replicate wells.

Example No. 6. FIG. 6 shows reduced ISG15 expression in breast cancer MCF-7 cells selected for low levels of CPT resistance. Panel A, formation of cleavable complexes in MCF7 and MCF7/RC breast cancer cells. Breast cancer MCF7 and MCF7/RC are treated with CPT (25 μM) for 15 min. Cells are then immediately lysed by the alkaline lysis procedure. Neutralized lysates are directly mixed with SDS sample buffer for loading onto a 6% SDS-polyacrylamide gel. Immunoblotting is performed with anti-hTOP1 I antibody. Panel B, CPT-induced TOP1 down-regulation in MCF7 and MCF7/RC cells. MCF7 and MCF7/RC cells are treated with either 1% DMSO (lanes #1 and 5) or CPT (25 μM in 1% DMSO) (lanes #2-5 and 6-8) for 0, 2, 4, and 6 hrs, followed by incubation in CPT-free medium for another 30 min to reverse TOP1-DNA covalent complexes. Cells are then lysed by the alkaline lysis procedure. Neutralized lysates are further treated with Staphylococcus nuclease S7 to release trapped TOP1 from residual TOP1-DNA covalent complexes. Immunoblotting is performed using anti-hTOP1 antibody. Panel C, the same membrane filter as in B is stripped and re-probed with anti-ISG15 antibody. Panel D, determination of IC50 by MTT assay. Cells are incubated with different concentrations of CPT continuously for 4 days. MTT assay is performed at least twice in six replicate wells.

Example No. 7. FIG. 8 shows a table showing CPT sensitivity for cells expressing different levels of ISG15. Cells are incubated with different concentrations of CPT continuously for 4 days. MTT assay is performed at least twice in six replicate wells.

Those skilled in the art will recognize that the above examples are illustrative of the present invention and not necessarily limiting thereto. Many other embodiments may be envisioned which are encompassed by the present invention, and the following claims.

What is claimed is:

1. A method for diagnosing cancer in a subject, comprising:
a. obtaining a sample from the subject;
b. determining the level of ISG15 in the sample using an assay; and
c. comparing the level of ISG15 with that of a standard;
wherein the presence of elevated ISG15 is indicative of the presence of cancer.

2. A method for determining the sensitivity of cancer to a DNA-damaging agent in a subject, comprising:
a. obtaining a sample from the subject;
b. determining the ISG15 level in the sample using an assay; and
c. comparing the level of ISG15 to that of a standard;
wherein the presence of elevated ISG15 serves as a biomarker and is indicative of the cancer's sensitivity to treatment with a DNA-damaging agent.

3. The method of claim 2, wherein the DNA-damaging agent is a topoisomerase I inhibitor.

4. A method of identifying an appropriate regimen for the treatment of cancer in a subject, comprising the steps of:
a. obtaining a sample from the subject;
b. determining the ISG15 level in the sample using an assay;
c. comparing the level of ISG15 to that of a standard;
d. Selecting DNA-damaging agents appropriate to the cancer; and
e. Optionally selecting additional chemotherapeutic agents for combination therapy.

5. The method of claim 4, wherein the DNA-damaging agent is a topoisomerase I inhibitor.

6. The method claim 4, wherein at least one of the chemotherapeutic agents is a molecule that upregulates ISG15 to enhance the sensitivity of the cancer to chemotherapy.

7. The method of claim 6, wherein the molecule is selected from the group consisting of all-trans retinoic acid, interferons, tumor necrosis factors, and mixtures thereof.

8. A method of monitoring a course of cancer treatment with chemotherapeutic agents, comprising the steps of:
a. obtaining a sample from the subject;
b. determining the ISG15 level in the sample using an assay; and
c. comparing the level of ISG15 to that of a standard.
d. Administering at least one chemotherapeutic agent to the subject;
e. Obtaining a second sample from the subject;
f. Measuring in the second sample the ISG15 level; and
g. Comparing the ISG15 level of the first sample with that of the second sample to determine changes in the sensitivity of the cancer to the chemotherapeutic agents.

9. The method of claim 8, wherein at least one of the chemotherapeutic agents is a topoisomerase I inhibitor.

10. The method of claim 9, wherein at least one of the chemotherapeutic agents is a small molecule that upregulates ISG15 expression.

11. The method of claim 9, wherein at least one of the chemotherapeutic agents is a biological molecule that upregulates ISG15.

12. The method of claim 11, wherein the biological molecule is selected from interferons, tumor necrosis factors and mixtures thereof.

13. The method of claim 1 or 2, wherein the sample is a blood sample.

14. The method of claim 13, wherein the sample is a member of the group consisting of serum, plasma, and whole blood and peripheral blood cells.

15. The method of claim 1 or 2, wherein the assay for determining the level of ISG15 is an immunoassay that detects the expression of ISG15.

16. The method of claim 1 or 2, wherein the assay for determining the level of ISG15 is a nucleic acid-based assay that detects the expression of messenger RNA.

17. The method claim 1 or 2, wherein the assay for determining the level of ISG15 detects other markers that correlate with the expression of ISG15.

18. The method of claim 15, wherein the detection is based on the use of monoclonal antibodies against the ISG15 protein.

19. The method of claim 15, wherein the detection is based on the use of polyclonal antibodies against the ISG protein.

20. The method of claim 2, wherein the method differentiates between benign and malignant tumors.

21. A method for the of treatment of cancer, comprising administering to a subject in need thereof a therapeutically effective amount of:
a. a topoisomerase I inhibitor; and
b. an agent that increases the expression of ISG15.

22. The method of claim 21, wherein the cancer expresses low levels of ISG15.

23. The method of claim 21, wherein the cancer expresses an elevated level of ISG15 but such a method is used to enhance the therapeutic effect.

24. The method of claim 21, wherein the agent that is used to enhance the expression of ISG15 is a small molecule.

25. The method of claim 24, wherein the small molecule is all-trans retinoic acid.

26. The method of claim 21, wherein the agent that is used to enhance the expression of ISG15 is a biological molecule.

27. The method of claim 26, wherein the biological molecule is an interferon.

28. The method of claim 26, wherein the biological molecule is a tumor necrosis factor.

29. A pharmaceutical composition for treating cancer, comprising
a. a therapeutically effective amount of a topoisomerase I inhibitor; and
b. a therapeutically effective amount of an agent that increases the expression of ISG15.

30. The composition of claim 29, wherein the topoisomerase I inhibitor is selected from the group consisting of camptothecin, irintotecan, topotecan, belotecan, 9-aminocamptotecan, 10-aminocamptotecan, 12-aminocamptotecan, 10,11-methylenedioxycamptotecan, 9-nitrocamptotecan, 9-nitro-10-hydroxycamptotecan, 10-hydroxycamptotecan, 11-formylcamtotecan, 10-chlorocamptotecan, 10-methylcamptotecan, and mixtures thereof.

31. The composition of claim 29, wherein the agent that increases the expression of ISG15 is selected from the group consisting of all-trans retinoic acid, an interferon, a tumor necrosis factor, or mixtures thereof.

32. A method for determining the sensitivity of cancer to a DNA-damaging agent in a subject, comprising:
a. obtaining a sample from the subject;
b. determining the E1, E2 and E3 levels in the sample using an assay; and
c. comparing the level of E1-E3 to that of a standard;
wherein the presence of elevated E1-E3 serves as a biomarker and is indicative of the cancer's sensitivity to treatment with a DNA-damaging agent.

33. A method for diagnosing cancer in a subject, comprising:
a. obtaining a sample from the subject;
b. determining the level of E1, E2 and E3 in the sample using an assay; and
c. comparing the level of E1-E3 with that of a standard;
wherein the presence of elevated E1-E3 is indicative of the presence of cancer.
